(19) United States
(12) Patent Application Publication
(10) Pub. No.: US 2023/0272417 A1
(43) Pub. Date: Aug. 31, 2023

(54) USE OF CIRCUTRN IN PREPARATION OF DRUG FOR TREATING HEART FAILURE, RECOMBINANT VECTOR, AND DRUG FOR TREATING HEART FAILURE

(71) Applicant: SHANGHAI UNIVERSITY, Shanghai (CN)

(72) Inventors: Junjie Xiao, Shanghai (CN); Lijun Wang, Shanghai (CN); Yuxue Hu, Shanghai (CN); Jiangpeng Sun, Shanghai (CN); Hongjian Chen, Shanghai (CN)

(21) Appl. No.: 17/837,248

(22) Filed: Jun. 10, 2022

(30) Foreign Application Priority Data
Feb. 25, 2022 (CN) 202210175224.0

(51) Int. Cl. C12N 15/86; A61K 31/713; C12N 15/11

(52) U.S. Cl. CPC C12N 15/86; A61K 31/713; C12N 15/111; C12N 2750/14143; C12N 2750/14122; C12N 2830/008

(57) ABSTRACT

The present disclosure relates to the technical field of biomedicine, in particular to use of circUTRN in preparation of a drug for treating heart failure, a recombinant vector, and a drug for treating heart failure. It is proved by experiments that the drug for treating heart failure prepared by the circUTRN is capable of treating or ameliorating the heart failure, and especially has a desirable therapeutic effect on the heart failure caused by ischemia-reperfusion injury (IRI). Specifically, the circUTRN is capable of improving cardiac systolic dysfunction induced by myocardial IRI 3 weeks. In addition, the circUTRN is also capable of reducing an infarct size of acute myocardial IRI; and overexpression of the circUTRN can inhibit cardiomyocyte apoptosis induced by oxygen-glucose deprivation/recovery.

### CROSS REFERENCE TO RELATED SEQUENCE LISTING

This application contains a computer readable form of a Sequence Listing in ACSII text format created on and electronically submitted via EFS-Web on Jun. 8, 2022. The size of the ACSII text file is 3,003 bytes and the file is incorporated herein by reference.

### CROSS REFERENCE TO RELATED APPLICATION

This application claims the benefit and takes priority of Chinese Patent Application No. 202210175224.0, filed on Feb. 25, 2022, the contents of which are herein incorporated by reference in its entirety as part of the present application.

### TECHNICAL FIELD

The present disclosure relates to the technical field of biomedicine, in particular to use of circUTRN in preparation of a drug for treating heart failure, a recombinant vector, and a drug for treating heart failure.

### BACKGROUND ART

Heart disease is extremely harmful, and almost all cardiovascular diseases eventually lead to heart failure. Myocardial injuries, caused by myocardial infarction, cardiomyopathy, hemodynamic overload, and inflammation, can cause changes in the myocardial structure and function, eventually resulting in poor ventricular pumping and/or filling function, which seriously threatens human life. Ischemic cardiomyopathy is one of the major diseases endangering national health in China, and timely reperfusion is a necessary procedure to save the ischemic heart. However, the reperfusion is generally accompanied by severe myocardial ischemia-reperfusion injury (IRI), and there is still no effective intervention in clinical practice. At present, the ischemic cardiomyopathy is mainly treated by thrombolytic therapy, interventional therapy and surgery clinically. However, the course of treatment generally causes various complications, such as bleeding, air embolism, puncture point bleeding, and infection.

Circular RNA (circRNA) is a special non-coding RNA; and different from traditional linear RNA (including a 5′-end and a 3′-end), the circRNA, due to a closed ring structure, is not affected by exoribonucleases, and has more stable expression without easy degradation. Functionally, the circRNAs can act as sponges for microRNAs or proteins, enabling regulation of downstream molecules and signaling pathways; some circRNAs further have an encoding potential, and can be translated into proteins or small peptides to function. Increasing evidences show that the circRNA plays an important regulatory role in the occurrence and development of diseases. In the cardiovascular field, studies have shown that circZNF292 promotes endothelial cell proliferation during myocardial development; circANRIL promotes apoptosis in atherosclerotic diseases; circHRCR inhibits the occurrence of pathological myocardial hypertrophy and heart failure by up-regulating a cardioprotective protein ARC; and circFOXO3 acts as a protein sponge in the cardiomyopathy, and regulates cardiac aging. Therefore, it is necessary to find a common regulatory gene related to IRI, such that the treatment of heart failure caused by the IRI can be widely realized.

### SUMMARY

To solve the above problems, the present disclosure provides use of circUTRN in preparation of a drug for treating heart failure, a recombinant vector, and a drug for treating heart failure. The drug for treating heart failure prepared by the circUTRN is capable of treating or ameliorating the heart failure, and especially has a desirable therapeutic effect on the heart failure caused by IRI.

To achieve the above objective, the present disclosure provides the following technical solutions.

The present disclosure provides use of circUTRN in preparation of a drug for treating heart failure, having a nucleotide sequence shown in SEQ ID NO: 1.

Preferably, the heart failure may be selected from the group consisting of acute heart failure and chronic heart failure.

The present disclosure further provides a recombinant vector expressing circUTRN, including circUTRN having a nucleotide sequence shown in SEQ ID NO: 1 and a cyclization vector.

Preferably, the cyclization vector may include a cytomegalovirus (CMV) promoter.

Preferably, the cyclization vector may be selected from the group consisting of a lentivirus overexpression vector and an adeno-associated virus overexpression vector.

Preferably, a circUTRN sequence may be located between EcoRI and NdeI restriction sites of the lentivirus overexpression vector.

Preferably, a circUTRN sequence may be located between EcoRI and BamHI restriction sites of an adeno-associated virus (AAV) overexpression vector.

The present disclosure further provides a drug for treating heart failure, including the recombinant vector.

Preferably, the drug may further be selected from the group consisting of a lentivirus vector system and an adeno-associated virus vector system.

Preferably, the heart failure may be selected from the group consisting of acute heart failure and chronic heart failure.

The present disclosure has the following beneficial effects:

The present disclosure provides use of circUTRN in preparation of a drug for treating heart failure, having a nucleotide sequence shown in SEQ ID NO: 1. It is proved by experiments that the drug for treating heart failure prepared by the circUTRN is capable of treating or ameliorating the heart failure, and especially has a desirable therapeutic effect on the heart failure caused by IRI. Specifically, the circUTRN is capable of improving cardiac systolic dysfunction induced by myocardial IRI for 3 weeks. In addition, the circUTRN is also capable of reducing an infarct size of acute myocardial IRI; and overexpression of the circUTRN can inhibit cardiomyocyte apoptosis induced by oxygen-glucose deprivation/recovery.

### BRIEF DESCRIPTION OF THE DRAWINGS

FIG. 1 shows that expression of circUTRN in myocardial tissues is significantly up-regulated after tail-vein injection of adeno-associated virus 9 (AAV9)-packaged circUTRN by fluorescence quantitative PCR;

FIGS. 2A-2C show that tail-vein injection of AAV9-packaged circUTRN improves myocardial IRI-induced cardiac systolic dysfunction for 3 weeks as detected by echocardiography;

FIGS. 3A-3C show that tail-vein injection of AAV9-packaged circUTRN reduces a severity of myocardial infarction caused by acute myocardial IRI; where AAR/LV: Area at risk/Left ventricle weight; INF/AAR: Infarct size/Area at risk; and

FIGS. 4A-4B show that the circUTRN can resist oxygen-glucose deprivation/recovery (OGD/R) model-induced apoptosis of neonatal mouse cardiomyocytes (n=6); EV means control vehicle; circUTRN means circUTRN overexpression vector; TUNEL positive indicates positive cardiomyocyte apoptosis; white arrows indicate cardiomyocyte apoptosis; α-Actinin, α-Actinin positive indicates that the cell is a cardiomyocyte; DAPI indicates the nucleus; where Sham means sham; IRI 3W means 3 weeks after IRI; AAV9-EV means AAV9 control virus; AAV9-OE-circUTRN means circUTRN overexpresses AAV9; *, P<0.05; **, P<0.01; and ***, P<0.001.

### DETAILED DESCRIPTION OF THE EMBODIMENTS

The present disclosure provides use of circUTRN in preparation of a drug for treating heart failure, having a nucleotide sequence shown in SEQ ID NO: 1:

5′-TGGATCTCTTAGAGCTGAATACGACGAATGAAG
TTTTCAAGCAGCACAAACTGAACCAAAATGATCAGC
TCCTGAGTGTCCCAGACGTCATCAACTGTCTGACCA
CCACTTACGATGGGCTTGAGCAGCTGCACAAGGACT
TGGTCAATGTTCCACTCTGCGTCGATATGTGTCTCA
ACTGGCTGCTCAACGTATACGACACGGGCCGGACTG
GAAAAATTCGGGTACAGAGTCTGAAGATTGGATTGA
TGTCTCTCTCCAAAGGCCTCTTAGAAGAGAAATACA
GATGTCTCTTTAAGGAGGTGGCAGGGCCAACAGAGA
TGTGTGACCAGCGGCAGCTTGGCCTGCTACTTCACG
ATGCCATCCAGATCCCTAGGCAGCTGGGGGAAGTAG
CAGCCTTTGGGGGCAGTAACATTGAGCCCAGTGTCC
GCAGCTGCTTCCAGCAGAATAACAACAAGCCAGAAA
TCAGTGTGAAGGAGTTTATAGACTGGATGCATTTGG
AACCCCAGTCCATGGTGTGGTTGCCGGTTCTGCATC
GGGTCGCAGCTGCTGAGACTGCAAAACATCAGGCCA
AATGCAACATCTGCAAAGAATGCCCGATTGTTGGGT
TCAGATACAGGAGCCTAAAGCATTTTAATTATGATG
TCTGCCAGAGTTGCTTCTTTTCTGGAAGAACAGCAA
AGGGCCACAAGTTACATTACCCGATGGTAGAATACT
GCATACCG-3′.

The circUTRN has a circAtlas ID of mmu-Utrn_0055. It is proved by experiments that the drug for treating heart failure prepared by the circUTRN is capable of treating or ameliorating the heart failure, and especially has a desirable therapeutic effect on the heart failure caused by IRI. Specifically, the circUTRN is capable of improving cardiac systolic dysfunction induced by myocardial IRI for 3 weeks. In addition, the circUTRN is also capable of reducing an infarct size of acute myocardial IRI; and overexpression of the circUTRN can inhibit cardiomyocyte apoptosis induced by oxygen-glucose deprivation/recovery.

In the present disclosure, the heart failure is preferably selected from the group consisting of acute heart failure and chronic heart failure, more preferably heart failure caused by IRI.

The present disclosure further provides a recombinant vector expressing circUTRN, including circUTRN having a nucleotide sequence shown in SEQ ID NO: 1 and a cyclization vector.

In the present disclosure, the cyclization vector preferably includes a CMV promoter, more preferably is selected from the group consisting of a lentivirus overexpression vector and an adeno-associated virus overexpression vector, and furthermore preferably a lentivirus overexpression vector pLO-ciR and an adeno-associated virus overexpression vector pK5ssAAV-ciR.

In the present disclosure, a circUTRN sequence is preferably located between EcoRI and NdeI restriction sites of the lentivirus overexpression vector. In the examples, the lentivirus overexpression vector pLO-ciR is preferably purchased from Guangzhou Geneseed Biotech Co., Ltd., with an item number of GS0103. The recombinant vector can express a target gene (with a sequence shown in SEQ ID NO: 1) as a circular RNA circUTRN in eukaryotic cells, thereby improving and/or treating heart failure.

In the present disclosure, a circUTRN sequence is preferably located between EcoRI and BamHI restriction sites of an AAV overexpression vector. In the examples, the AAV overexpression vector pK5ssAAV-ciR is preferably purchased from Guangzhou Geneseed Biotech Co., Ltd., with an item number of GS0109. The recombinant vector can express a target gene (with a sequence shown in SEQ ID NO: 1) as a circular RNA circUTRN in eukaryotic cells, thereby improving and/or treating heart failure.

The present disclosure further provides a drug for treating heart failure, including the recombinant vector. When the cyclization vector of the recombinant vector is an AAV overexpression vector, a preparation method of the drug includes preferably AAV packaging; and the AVV includes preferably AAV9. There is no special requirement on packaging of the drug, and operation methods well-known to those skilled in the art can be used. The AAV does not participate in the occurrence of any disease, has low immunogenicity, and can continuously express genes for more than half a year; where the AAV9 has a strong affinity for cardiac tissues, and can be used as a vector to stably and efficiently express a target gene sequence in the cardiac tissues.

In the present disclosure, when the cyclization vector of the recombinant vector is a lentivirus overexpression vector, a preparation method of the drug includes preferably lentivirus packaging. There is no special requirement on packaging of the drug, and operation methods well-known to those skilled in the art can be used.

In the present disclosure, the drug further includes preferably a pharmaceutically acceptable auxiliary material.

In the present disclosure, the heart failure is preferably selected from the group consisting of acute heart failure and chronic heart failure, more preferably heart failure caused by IRI.

In order to further illustrate the present disclosure, the use of circUTRN in preparation of a drug for treating heart failure, the recombinant vector, and the drug for treating heart failure provided by the present disclosure are described in detail below in connection with examples, but these examples should not be understood as limiting the claimed scope of the present disclosure.

### EXAMPLE 1

A recombinant vector expressing circUTRN was constructed by the following steps:
1) A total RNA of a mouse heart tissue was reverse transcribed to cDNA, followed by PCR amplification, double-enzyme digestion, and DNA sequence of circUTRN was recovered (as shown in SEQ ID NO.1); where an upstream primer of the PCR amplification had a nucleotide sequence shown in SEQ ID NO: 2:

5′-CGGAATTCTGAAATATGCTATCTTACAGT
GGATCTCTTAGAGCTGAATACGACG-3′;

and a downstream primer of the PCR amplification had a nucleotide sequence shown in SEQ ID NO.3:

5′-GGAATTCCATATGTCAAGAAAAAATATAT
TCACCGGTATGCAGTATTCTACCATCGG-3′;

Each 50 μL of a PCR amplification reaction system included the following components: 1 μL of an upstream primer (10 μM) (at a final concentration of 0.2 μM), 1 μL of a downstream primer (10 μM) (at a final concentration of 0.2 μM), 25 μL of a 2× TransStart FastPfu PCR SuperMix (TransGen Biotech, Cat. No. AS221), 1 μL of a cDNA template (500 ng/μL), and 22 μL of enzyme-free water.

A PCR reaction included: initial denaturation at 95° C. for 2 min; denaturation at 95° C. for 20 sec, annealing at 58° C. for 20 sec, and extension at 72° C. for 30 sec, conducting 36 cycles; and heat preservation at 4° C.

A double-enzyme digestion system included: 1 μL of EcoRI-HF (NEB), 1 μL of NdeI (NEB), 5 μL of a 10× CutSmart buffer, 1 μg of a PCR product/or vector, and enzyme-free water (diluting to 50 μL).

A double-enzyme digestion reaction was conducted by: at 37° C. for 4 h.

A PCR product after double-enzyme digestion was cleaned and recovered using an ultra-thin DNA product purification kit (TIANGEN Biotech (Beijing) Co., Ltd., Cat. No. DP203).
2) After double-enzyme digestion (a method was the same as step 1) was conducted on the cyclization vector (a lentivirus overexpression vector pLO-ciR, purchased from Guangzhou Geneseed Biotech Co., Ltd., with an item number of GS0103), large fragment vectors were recovered using an ultra-thin agarose gel DNA recovery kit (TIANGEN Biotech (Beijing) Co., Ltd., Cat. No. DP208).
3) The fragments recovered in step 1) were inserted into the cyclization vector recovered in step 2) to obtain a ligation product. A total volume of a specific reaction system was 10 μL, including: 1 μL of a T4 DNA ligase, 1 μL of a 10× T4 DNA ligase buffer, 2 μL of a double-enzyme digested vector (100 ng), 1 μL of a double-enzyme digested gene (100 ng), and 5 μL of enzyme-free water.

The reaction was conducted by: at 16° C. for 8 h.
4) 50 μL of competent cells (competent cells were TransStbl3 Chemically Competent Cells, TransGen Biotech, Cat. No. CD521) were dissolved on ice, and 5 μL of the ligation product prepared in step 3) was added, an obtained mixture was placed on the ice for 25 min after flicking to mix well; heat shock was conducted at 42° C. for 45 sec, and the mixture was placed back on the ice for 2 min; 500 μL of a medium (SOC or LB medium, TransGen Biotech, Cat#CD521) was added, and after mixing, incubation was conducted at 37° C. for 1 h at 180 r/min to revive the bacteria. The bacteria were plated on an ampicillin-resistant plate and incubated overnight. The monoclonal shaken bacteria were sent to Sanger for sequencing.

### EXAMPLE 2

A recombinant vector expressing circUTRN similar to Example 1, where differences were as follows:
1, a downstream primer in step 1) was replaced with a downstream primer having a nucleotide sequence shown in SEQ ID NO: 4, as follows:
5′-CGGGATCCAGTTGTTCTTACCGGTATGCAGTATTCTACCATCGG-3′; 2, NdeI (NEB) in step 1) was replaced with BamHI-HF (NEB); and
3, a cyclization vector in step 2) was replaced with an AAV overexpression vector pK5ssAAV-ciR (purchased from Guangzhou Geneseed Biotech Co., Ltd., with an item number of GS0109).

### EXAMPLE 3

A preparation method of a drug for treating heart failure included the following steps:

293T cells were added to a packaging system at a suitable cell density (80%); each 10 cm of a dish was added with 1 mL of the packaging system, including: 10 μg of AAV2/9 (purchased from Addgene Plasmid, Catalog #112865), 10 μg of pAdDeltaF6 plasmid (purchased from Addgene Plasmid, Catalog #112867), and 10 μg of the recombinant vector constructed in Example 2; DMEM medium (no FBS or penicillin-streptomycin solution) was added to dilute to 910 μL, and 90 μL of a PEI transfection reagent was added (a total volume was: 1 mL); after 60 h of cell transfection, a supernatant and its cells were collected; the virus was concentrated with iodixanol by ultracentrifugation (the collected supernatant was centrifuged and concentrated to 10 mL to 15 mL using a concentration column (Merck UFC905096) at 4,000 rpm and 4° C.), and an obtained virus suspension was the drug; where the virus suspension had a titer of 1×10¹³ vg/mL.

### USE EXAMPLE 1

Establishment of a myocardial IRI model: the IRI model adopted the most severe injury effect, namely, myocardial ischemia was caused by ligation of a left anterior descending coronary artery for 30 min, and reperfusion was conducted. Briefly, wild-type male mice of 8-10 weeks old were injected intraperitoneally with 4% chloral hydrate at a dosage of 10 μl/g for anesthesia. After anesthesia, the mice were fixed on a thermostat blanket with their abdomen facing up using a medical tape, and the hairs on the back neck were removed with depilatory cream. The hair removal part were sterilized with 75% ethanol; under a microscope, a horizontal incision was made at the fourth and fifth intercostal space at a left sternum of the mouse using small scissors, where the incision was about 1.2 cm; chest wall muscles were separated layer by layer until intercostal muscles were exposed, and the intercostal muscles were bluntly separated using microscopic forceps to expose the heart; the left anterior descending artery was ligated (since the space between a left atrial appendage and a pulmonary artery cone was invisible to the naked eyes, the success of ligation depended on the ischemia (whitening) of the underlying cardiac apex). The intercostal muscles and chest wall muscles were sutured. After 30 min, the chest cavity was opened again, and a ligature was cut and removed. The mice were kept warm on a constant-temperature blanket, and after recovery, the mice were transferred to mouse cages for 24 h OM acute model) or 21 d (IRI 3 weeks, long-term remodeling model); the mice were sacrificed, body weight, tibia length, and heart weight of the mice were measured, and samples were retained for subsequent detection. The sham in this example was similar to the above IRI operation, the only difference was that no ligation was conducted.

The experiment was divided into 4 groups, specifically as follows:
A first group was a control virus (AVV2/9 in Example 3)+sham group. A control virus was administrated to mice via tail-vein injection, all at 10^¹¹ vg/mouse, and the sham was conducted one week later. There were 10 samples.
A second group was AAV9-circUTRN virus (drug prepared in Example 3)+sham group. An AAV9-circUTRN virus was administrated to mice via tail-vein injection, all at 10^¹¹ vg/mouse, and the sham was conducted one week later. There were 10 samples.
A third group was a control virus (AVV2/9 in Example 3)+IRI operation group. A control virus was administrated to mice via tail-vein injection, all at 10^¹¹ vg/mouse, and the IRI operation was conducted one week later. There were 14 samples.
A fourth group was a AAV9-circUTRN virus (drug prepared in Example 3)+IRI operation group. An AAV9-circUTRN virus was administrated to mice via tail-vein injection, all at 10^¹¹ vg/mouse, and the IRI operation was conducted one week later. There were 13 samples.

A total RNA was extracted from the mouse heart tissue of the above four groups using RNAiso Plus, and a relative expression level of circUTRN three weeks after sham and TM operation were detected by real time fluorescence quantitative PCR.

The details were as follows: 18s was used as an internal reference gene (specific sequences were F1: 5′-TCAAGAACGAAAGTCGGAGG-3′, SEQ ID NO: 5 and R1: 5′-GGACATCTAAGGGCATCAC-3′, SEQ ID NO: 6); qPCR primer sequences of circUTRN were: F2: 5′-GGCCACAAGTTACATTACCCG-3′, SEQ ID NO: 7 and R2: 5′-acgttgagcagccagttgag-3′, SEQ ID NO: 8; results of the qPCR were analyzed by a relative quantitative method, and calculated using 2^{-ΔΔCt}, as follows: ΔCt=Ct value of target gene−Ct value of internal reference, ΔΔCt=ΔCt value of each experimental group−average ΔCt value of control group.

A specific method of real-time fluorescence PCR (10 μL system) included:
Preparation of a primer working solution: a stock solution (100 μM) was diluted 20 times to obtain a mother liquor (5 μM), and the mother liquor was diluted 10 times to obtain the working solution (0.5 μM).
Preparation of a mixture of cDNA and SYBR: a common volume ratio was cDNA: SYBR=1:20 (5 μL/well).
Plate adding: 5 μL of the primer working solution was added, and 5 μL of the mixture of cDNA and SYBR was added to form a system of 10 μL.
PCR reaction program: BIO-RAD 2 STEP
Initial denaturation at 95° C. for 30 sec; denaturation at 95° C. for 15 sec, and annealing at 60° C. for 60 sec, conducting 40 cycles;
Melting curve analysis: at 95° C. for 1 sec, at 60° C. for 1 sec, at 95° C. continuous
(65° C. to 95° C. 0.5° C. increments at 2-5 sec/step)
Cooling 40° C. 30 sec.

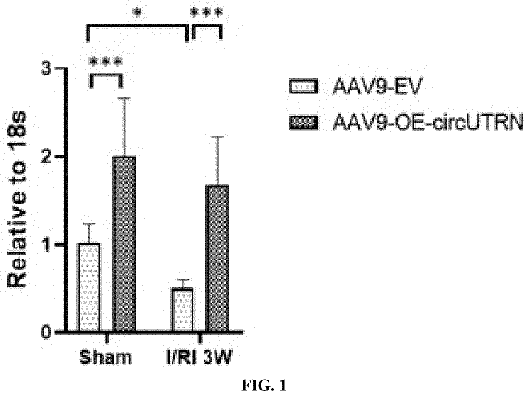

Specifically, relative RNA contents of 10 mice in the first group were 0.838568, 1.141555, 1.018185, 1.334223, 0.75576, 0.835667, 0.777007, 1.236275, 1.249196, and 1.014663, respectively;
relative RNA contents of 10 mice in the second group were 1.848045, 1.522033, 1.718322, 1.470187, 2.940375, 2.631709, 3.108029, 1.712377, 1.913216, and 1.181812, respectively;
relative RNA contents of 14 mice in the third group were 0.454074, 0.753145, 0.557097, 0.503827, 0.541863, 0.491751, 0.560972, 0.592957, 0.484981, 0.473357, 0.368823, 0.410655, 0.373971, 0.483303, respectively; and
relative RNA contents of 13 mice in the fourth group were 1.83528, 1.575708, 2.167452, 1.223488, 1.516768, 2.613531, 2.455471, 1.53262, 0.796088, 1.454981, 1.642621, 2.057653, and 0.911301, respectively. The statistical results are shown in FIG. 1, where *, P<0.05, ***, P<0.001.

The results show that the expression of circUTRN is down-regulated three weeks after IRI operation; after tail-vein injection of AAV9-packaged circUTRN, the expression of circUTRN is significantly up-regulated in cardiac tissues.

### USE EXAMPLE 2

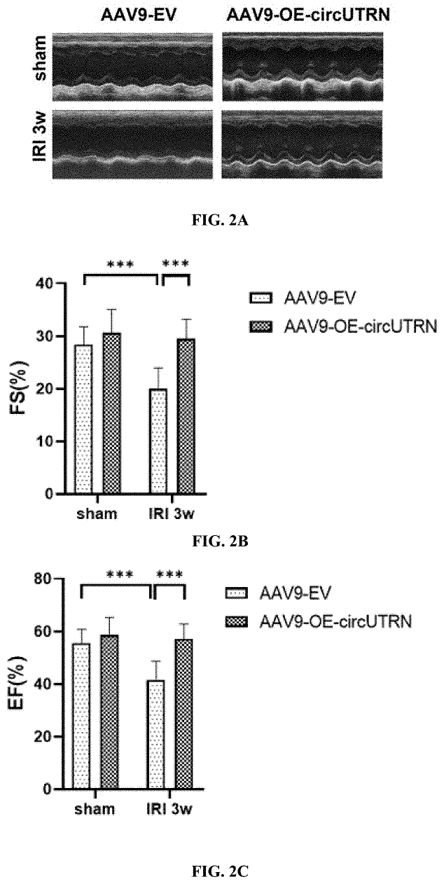

By the same grouping and treating methods as those in Use Example 1, C57BL/6J adult male mice were selected for ligation of the left anterior descending coronary artery, and the ligation was released after 30 min; three weeks after IRI operation, echocardiography was conducted to measure cardiac function-related indices (ejection fraction (EF) and fractional shortening (FS)) in the above four groups. Cardiac function of mice were measured by using Vevo 2100 echocardiography: the chest of the mice was depilated, and the mice were anesthetized with isoflurane inhalation anesthesia; limbs of the mouse were fixed on an ultrasound plate, and an appropriate amount of chelating agent was applied to the abdominal heart of the mouse; When the heart rate was stable at around 400 bpm, parameters were measured from M-mode images taken from the parasternal short-axis view at papillary muscle level, and the cardiac function was measured and calculated, to obtain the cardiac function-related parameters: the left ventricular EF and the left ventricular FS. The results were shown in FIGS. 2A-2C.

Specifically, FS(%) of 10 mice in the first group were 26.57607, 28.73468, 30.00613, 34.14948, 32.35228, 29.93588, 22.36367, 26.9502, 27.2448, and 26.21533, respectively;

FS(%) of 10 mice in the second group were 28.37145, 34.25649, 31.288, 26.87928, 31.41054, 37.83692, 27.23812, 22.8164, 34.70449, and 32.0555, respectively;

FS(%) of 14 mice in the third group were 25.7799, 25.72851, 17.9281, 16.89639, 16.79336, 18.24466, 17.54019, 20.96864, 16.8033, 23.96844, 14.63545, 24.6609, 16.61429, and 23.86121, respectively; and

FS(%) of 13 mice in the fourth group were 31.41518, 30.00557, 30.17961, 28.22829, 27.84015, 34.28185, 34.86179, 29.70271, 28.46201, 22.60534, 34.172137, 27.64563, and 24.13351, respectively.

Specifically, EF(%) of 10 mice in the first group were 52.58755, 55.87751, 58.14579, 64.05795, 61.45018, 57.95299, 45.50572, 53.6604, 53.85443, and 52.0395, respectively;

EF(%) of 10 mice in the second group were 55.51394, 64.23814, 59.70567, 53.01966, 60.23159, 68.61732, 53.49452, 46.05206, 65.00643, and 60.71237, respectively;

EF(%) of 14 mice in the third group were 51.63186, 51.77215, 37.77709, 35.63613, 35.49855, 38.65654, 37.26884, 43.38542, 35.61092, 48.38518, 32.21303, 49.7293, 35.34495, and 49.15511, respectively; and

EF(%) of 13 mice in the fourth group were 60.09334, 57.32902, 58.13966, 55.29248, 54.55704, 64.67837, 65.48337, 57.59445, 56.06144, 46.36027, 63.965966, 54.38887, and 49.07793, respectively. The statistical results are shown in FIGS. 2A-2C, where **, P<0.01.

As shown in FIGS. 2A-2C, tail-vein injection of circUTRN overexpression AAV9 can improve myocardial IRI 3 weeks-induced cardiac systolic dysfunction.

### USE EXAMPLE 3

A 2,3,5-triphenyltetrazolium chloride (TTC) staining experiment was conducted, and divided into 2 groups with 10 mice in each group, specifically as follows:
Group 1: control AAV9 (the AVV2/9 in Example 3) was injected through the tail vein with a disposable 1 mL sterile syringe starting 1 week before the establishment of the IRI model, at a dose of 10′ vg/mouse.
Group 2: AAV9-circUTRN (the drug prepared in Example 3) was injected through the tail vein with a disposable 1 mL sterile syringe starting 1 week before the establishment of the IRI model, at a dose of 10′ vg/mouse. One week later, the two groups underwent IRI operation using the method in Use Example 1, and samples were taken 24 h later.

TTC staining: the mice after IRI operation were anesthetized with 4% chloral hydrate intraperitoneally, at 200 μL per 20 g of body weight of the mice. After being anesthetized, the mice were laid with its abdomen up and fixed on a foam board, and the chest skin, muscles and ribs were cut with scissors to expose the heart. A heart ligation site during ischemic surgery was re-ligated with a 7-0 needle; 1 mL to 2 mL of 1% Evans Blue was aspirated with a 1 mL insulin syringe, and injected into the left ventricle of the mouse heart; the mouse nose tips and extremities were observed, and the injection was terminated until the nose tips and extremities turned blue. After removing the heart, the heart was placed in a 1.5 mL centrifuge tube and temporarily stored at −20° C. The heart was sliced after freezing and hardening. The sliced heart was put in a groove of a slicing mold, and blades were inserted into a space from top to bottom through a surface of the heart according to the space of the groove. After being were fully inserted, the blades were pressed in parallel to cut the heart into 1 mm pieces, the blades were removed, and the heart slices were put into a 1.5 mL centrifuge tube. A pre-prepared 1% TTC solution was added to the centrifuge tube, at 1 mL per tube, and the centrifuge tube was placed in a water bath at 37° C. for 10 min in the dark. A 4% PFA solution was added to a 96-well plate for fixation of the heart slices. After the water bath, the heart slices were taken out of the centrifuge tube and placed in the 96-well plate for fixation, with one slice per well. The fixation was conducted for 1.5 h. Pictures were taken 1.5 h after the slices were fixated to prevent color loss over time. Each piece of heart in the 96-well plate was weighed and recorded. Statistics was conducted on front whole slice area, front white infarct area, and front red area, as well as back whole slice area, back white infarct area, and back red area of the heart slices by manually circling using Image J software. The table was filled with the area data corresponding to the weight of each slice.

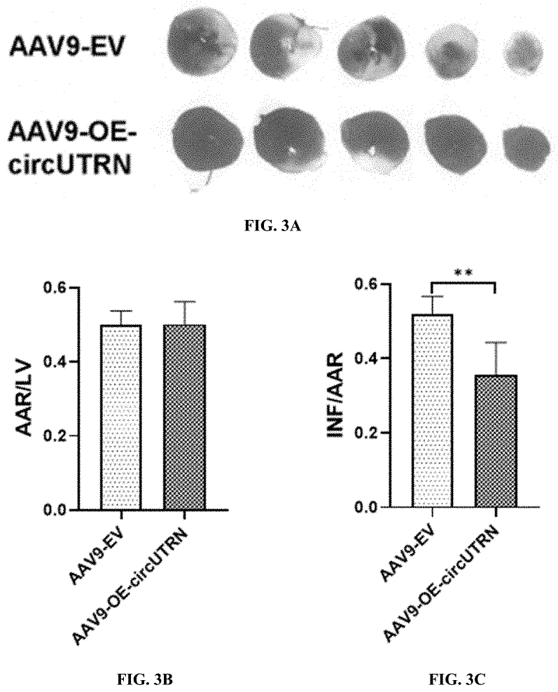

The experimental results are shown in FIGS. 3A-3C. Specifically, the AAR/LV ratios of group 1 were 0.546797124, 0.498411923, 0.573749793, 0.478128607, 0.502519571, 0.474636482, 0.466649783, 0.501469873, 0.504364335, and 0.445463737, respectively;
the AAR/LV ratios of the first group were 0.420656141, 0.501991857, 0.504419175, 0.412981739, 0.596882045, 0.560244519, 0.55786186, 0.475217199, 0.523069062, and 0.459159742, respectively;
the INF/AAR ratios of the first group were 0.412583, 0.558483, 0.544388, 0.485926, 0.573036, 0.541956, 0.484268, 0.498673, 0.548814, and 0.542946, respectively; and
the INF/AAR ratios of the second group were 0.278354675, 0.261470747, 0.473479359, 0.387369342, 0.245335, 0.367833474, 0.349436683, 0.497779168, 0.404129982, and 0.296345906, respectively. The statistical results are shown in FIGS. 3B-3C, where **, P<0.01.

The results show that tail-vein injection of circUTRN overexpression AAV9 can reduce the myocardial infarction size which was caused by myocardial IRI.

### USE EXAMPLE 4

Establishment of cardiomyocyte OGD/R model and plasmid transfection: NMCM was cultured with a normal cardiomyocyte medium, and when NMCMs were spread to 80% of a well area, a serum-free medium was changed to starvation for 7 h; 6 μL of Lipofectamine 2000 was added to 100 μL of a serum-free DMEM, premixed for 5 min (mixed gently and then let stand for 5 min), to obtain solution A; 2 μg of a plasmid was added to 100 μL of the serum-free DMEM, premixed for 5 min (mixed gently and then let stand for 5 min), to obtain solution B; after premixing, the solution A and solution B were mixed, and after standing at room temperature for 20 min, a mixture was added at 100 μL/well to a 96-well plate; after 7 h of transfection, a transfection medium was discarded and replaced with the serum-free medium, and the cells were continued to be incubated in a constant-temperature incubator at 37° C., 5% CO₂; after changing the medium for 20 h, the medium of the cells was changed to no glucose DMEM, and the cell culture plate was placed in an air-tight chamber with a humidified hypoxic atmosphere to conduct exposure to oxygen glucose deprivation assay, after incubation for 8 h; and the cell culture plate was removed from the hypoxia box for reoxygenation, and the no glucose DMEM was changed back to the normal cardiomyocyte medium to further incubation for 12 h, to the end of the experiment.

The recombinant vector (0.02 mg/L) overexpressing circUTRN prepared in Example 1 was transfected into neonatal mouse cardiomyocytes, and the cells were collected 72 h after transfection; the OGD/R model was constructed 20 h before the end of experiment (8 h of hypoxia and glucose deprivation, and 12 h of reoxygenation and glucose recovery), such that the gain-of-function experiment was completed. The specific groups were: 1) circUTRN control group (the lentivirus empty vector pLO-ciR in Example 1), 2) circUTRN control+OGD/R treatment group, 3) circUTRN overexpression group (the recombinant vector overexpressing circUTRN prepared in Example 1), and 4) circUTRN overexpression+OGD/R treatment group. At the end of experiment, a protective effect of circUTRN on the cardiomyocyte apoptosis was detected by immunofluorescence co-staining of Tunel and α-actinin/DAPI.

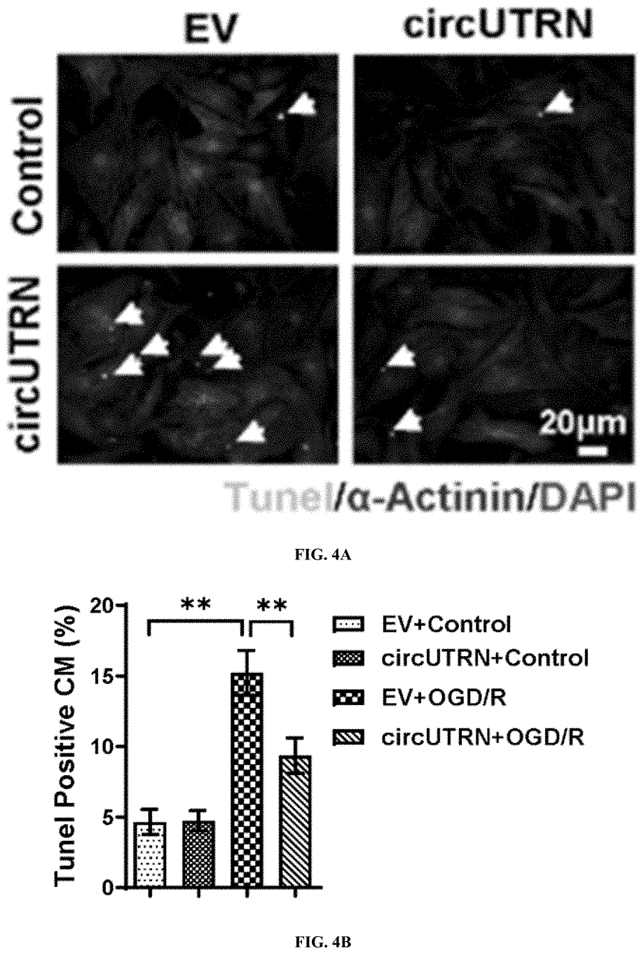

It can be seen from FIGS. 4A-4B that in the OGD/R model, the apoptosis level of NMCM is significantly increased compared with the control group; moreover, the overexpression of circUTRN at a basal level has no effect on the apoptosis; however, under OGD/R stimulation, the overexpression of circUTRN can protect NMCM from apoptosis induced by OGD/R. Specifically, the apoptosis rates (%) of group 1) were 4.164398, 3.660825, 3.954054, 5.02763, 5.036923, and 6.062882, respectively;
the apoptosis rates (%) of group 2) were 4.35287, 5.562385, 4.304403, 5.45163, 3.801478, and 4.991256, respectively;
the apoptosis rates (%) of group 3) were 12.97532, 15.21066, 15.17345, 17.85742, 14.54959, and 15.53715, respectively; and

Specifically, the apoptosis rates (%) of group 4) were 9.172941, 9.148951, 8.449327, 11.39639, 7.821497, and 10.04349, respectively. The statistical results are shown in FIG. 4B, where **, P<0.01.

In summary, the circUTRN is capable of improving cardiac systolic dysfunction induced by myocardial IRI 3 weeks. In addition, the circUTRN is also capable of reducing an infarct size of acute myocardial IRI; and overexpression of the circUTRN can inhibit cardiomyocyte apoptosis induced by oxygen-glucose deprivation/recovery.

The present disclosure has been disclosed with preferred examples as above, which shall not be construed as a limitation to the present disclosure. Any person skilled in the art can make changes and variations without departing from the spirit and scope of the present disclosure. The present disclosure shall fall within the protection scope defined in the claims.

What is claimed is:

1. Use of circUTRN in preparation of a drug for treating heart failure, having a nucleotide sequence shown in SEQ ID NO: 1.

2. The use according to claim 1, wherein the heart failure is selected from the group consisting of acute heart failure and chronic heart failure.

3. A recombinant vector expressing circUTRN, comprising circUTRN having a nucleotide sequence shown in SEQ ID NO: 1 and a cyclization vector.

4. The recombinant vector according to claim 3, wherein the cyclization vector comprises a cytomegalovirus (CMV) promoter.

5. The recombinant vector according to claim 3, wherein the cyclization vector is selected from the group consisting of a lentivirus overexpression vector and an adeno-associated virus overexpression vector.

6. The recombinant vector according to claim 4, wherein the cyclization vector is selected from the group consisting of a lentivirus overexpression vector and an adeno-associated virus overexpression vector.

7. The recombinant vector according to claim 5, wherein a circUTRN sequence is located between EcoRI and NdeI restriction sites of the lentivirus overexpression vector.

8. The recombinant vector according to claim 6, wherein a circUTRN sequence is located between EcoRI and NdeI restriction sites of the lentivirus overexpression vector.

9. The recombinant vector according to claim 5, wherein a circUTRN sequence is located between EcoRI and BamHI restriction sites of an adeno-associated virus (AAV) overexpression vector.

10. The recombinant vector according to claim 6, wherein a circUTRN sequence is located between EcoRI and BamHI restriction sites of an adeno-associated virus (AAV) overexpression vector.

11. A drug for treating heart failure, comprising the recombinant vector according to claim 3.

12. The drug according to claim 11, wherein the cyclization vector comprises a cytomegalovirus (CMV) promoter.

13. The drug according to claim 11, wherein the cyclization vector is selected from the group consisting of a lentivirus overexpression vector and an adeno-associated virus overexpression vector.

14. The drug according to claim 12, wherein the cyclization vector is selected from the group consisting of a lentivirus overexpression vector and an adeno-associated virus overexpression vector.

15. The drug according to claim 13, wherein a circUTRN sequence is located between EcoRI and NdeI restriction sites of the lentivirus overexpression vector.

16. The drug according to claim 14, wherein a circUTRN sequence is located between EcoRI and NdeI restriction sites of the lentivirus overexpression vector.

17. The recombinant vector according to claim 13, wherein a circUTRN sequence is located between EcoRI and BamHI restriction sites of an adeno-associated virus (AAV) overexpression vector.

18. The drug according to claim 11, wherein a preparation method of the drug comprises virus packaging.

19. The drug according to claim 11, wherein the heart failure is selected from the group consisting of acute heart failure and chronic heart failure.

20. The drug according to claim 18, wherein the heart failure is selected from the group consisting of acute heart failure and chronic heart failure.


SEQUENCE LISTING (8 sequences)

SEQ ID NO: 1 - DNA, 725 bp, Artificial Sequence
  circUTRN
tggatctcttagagctgaatacgacgaatgaagttttcaagcagcacaaactgaaccaaaatgatcagctcctgagtgtcccagacgtcatcaactgtctgaccaccacttacgatgggcttgagcagctgcacaaggacttggtcaatgttccactctgcgtcgatatgtgtctcaactggctgctcaacgtatacgacacgggccggactggaaaaattcgggtacagagtctgaagattggattgatgtctctctccaaaggcctcttagaagagaaatacagatgtctctttaaggaggtggcagggccaacagagatgtgtgaccagcggcagcttggcctgctacttcacgatgccatccagatccctaggcagctgggggaagtagcagcctttgggggcagtaacattgagcccagtgtccgcagctgcttccagcagaataacaacaagccagaaatcagtgtgaaggagtttatagactggatgcatttggaaccccagtccatggtgtggttgccggttctgcatcgggtcgcagctgctgagactgcaaaacatcaggccaaatgcaacatctgcaaagaatgcccgattgttgggttcagatacaggagcctaaagcattttaattatgatgtctgccagagttgcttcttttctggaagaacagcaaagggccacaagttacattacccgatggtagaatactgcataccg

SEQ ID NO: 2 - DNA, 54 bp, Artificial Sequence
  upstream primer for PCR of recombinant vector expressing circUTRN
cggaattctgaaatatgctatcttacagtggatctcttagagctgaatacgacg

SEQ ID NO: 3 - DNA, 57 bp, Artificial Sequence
  downstream primer for PCR of recombinant vector expressing circUTRN
ggaattccatatgtcaagaaaaaatatattcaccggtatgcagtattctaccatcgg

SEQ ID NO: 4 - DNA, 44 bp, Artificial Sequence
  downstream primer for PCR of similar recombinant vector expressing circUTRN
cgggatccagttgttcttaccggtatgcagtattctaccatcgg

SEQ ID NO: 5 - DNA, 20 bp, Artificial Sequence
  internal reference primer F1
tcaagaacgaaagtcggagg

SEQ ID NO: 6 - DNA, 19 bp, Artificial Sequence
  internal reference primer R1
ggacatctaagggcatcac

SEQ ID NO: 7 - DNA, 21 bp, Artificial Sequence
  qPCR primer sequences of circUTRN F2
ggccacaagttacattacccg

SEQ ID NO: 8 - DNA, 20 bp, Artificial Sequence
  qPCR primer sequences of circUTRN R2
acgttgagcagccagttgag